(19) United States
(12) Patent Application Publication
(10) Pub. No.: US 2016/0008531 A1
(43) Pub. Date: Jan. 14, 2016

(54) DUAL LUMEN CANNULA FOR ARTIFICIAL LUNG AND RIGHT VENTRICULAR ASSIST DEVICE

(71) Applicant: W-Z Biotech, LLC, Lexington, KY (US)

(72) Inventors: Dongfang Wang, Lexington, KY (US); Joseph B. Zwischenberger, Lexington, KY (US)

(21) Appl. No.: 14/635,105

(22) Filed: Mar. 2, 2015

Related U.S. Application Data
(63) Continuation-in-part of application No. 12/539,110, filed on Aug. 11, 2009.

(51) Int. Cl. A61M 1/36; A61M 1/32; A61M 1/10

(52) U.S. Cl. CPC A61M 1/3659; A61M 1/1008; A61M 1/32

(57) ABSTRACT

A dual lumen cannula includes a first lumen having at least one aperture for removing a fluid from the interior of the heart right atrium, and a second lumen for delivering a fluid into the interior of the pulmonary artery or the heart left atrium. The second lumen includes a distal selectively curvable portion having a length of from about 4 cm to about 22 cm to position an end thereof within the interior of the pulmonary artery lumen or the human heart left atrium interior when a distal end of the first lumen is positioned within a right atrium of the human heart. The second lumen may also include a distal anchoring structure for preventing inadvertent withdrawal from a surgical site.

This utility patent application is a continuation-in-part of U.S. patent application Ser. No. 12/539,110 filed on Aug. 11, 2009, the entirety of the disclosure of which is incorporated herein by reference.

### TECHNICAL FIELD

The present invention relates generally to the field of cardiac medical devices and systems. In particular, the invention relates to a dual lumen cannula for use in artificial lung (AL) and right ventricular assist device (RVAD) applications.

### BACKGROUND OF THE INVENTION

It is known to provide assemblies for removing blood from a patient for oxygenation and removal of carbon dioxide via an artificial lung, and/or passing blood through an artificial heart-like device such as a right ventricular assist device to support the pumping function of a failing heart. Such assemblies utilize various cannula designs to remove oxygen-depleted blood from the patient and to return oxygenated blood to the patient. In particular, it is known to provide various designs of dual lumen cannulae, whereby oxygen-depleted blood is removed via the first lumen, and oxygenated blood is returned via the second lumen. Conventionally, cannulae for use in such blood access assemblies during extracorporeal blood oxygenation and like procedures typically are implanted in a peripheral blood vessel such as the jugular vein.

Disadvantageously, such systems are unsuited for long term, repeated use. Rather, during use, patient mobility is restricted and at the end of a treatment session, the cannula must be removed. In the event of subsequent treatment sessions, another cannula must be implanted, requiring multiple instances of cannula implantation and removal, with associated discomfort, trauma, and potential issues of infection to the patient. Thus, there remains a need in the art for novel devices, and methods and systems incorporating them, for providing routine, repeated access to an interior of the heart for procedures such as right ventricular assist procedures, artificial lung procedures, and the like, provided over a longer time period.

Alternatively, there are situations wherein access through the heart via the chest wall is not desirable. For example, such a surgical procedure may represent an untenable risk to a particular patient. Further, there are situations wherein an extreme surgical procedure such as accessing the heart via the chest wall, while one potential option, is undesirable due to the short-term nature of the problem to be solved. As an example, in the event of left heart failure, a left heart assist device may be implanted to provide support to one or more of the left chambers of the heart. In such situations, it is common, once the problem with the left side of the heart is ameliorated, for the right side of the heart to experience failure. This tends to occur one or two days after the left heart procedure is completed. Typically, with use of an RVAD, the problem with the right side of the heart can be corrected in approximately a week. In such situations, while it is possible to open the patient's chest to install a support device such as an RVAD, the surgery is extensive and requires extended recovery time. In such situations where providing short-term assistance to the blood pumping function of the heart is needed, percutaneous access to the heart interior may be more desirable. The present disclosure provides also improved cannula designs for accomplishing this goal.

### SUMMARY OF THE INVENTION

In one aspect of the present disclosure there is described a dual lumen cannula including a first lumen having a first length and including at least one distal drainage aperture and a second lumen having a length that is greater than the first lumen length, a selectively curvable portion for assuming a predetermined angle or curvature, and at least one distal infusion aperture.

The second lumen selectively curvable portion in embodiments has a length dimension whereby the second lumen at least one infusion aperture is disposed a distance of from about 4 cm to about 22 cm from the first lumen drainage aperture to allow positioning the second lumen infusion aperture in an interior of a pulmonary artery lumen or a left atrium of the human heart when the first lumen distal drainage aperture is positioned within a right atrium of the human heart. In embodiments, the distal drainage aperture includes a closure that is selectively translatable from a first, closed position to a second, open position. The predetermined angle or curvature in the selectively curvable portion may be created in embodiments by at least one of a spring elbow, a flexible polymer, or a shape memory alloy. The second lumen may optionally include a distal anchoring structure for preventing inadvertent withdrawal of the second lumen end from a surgical site. In embodiments, the anchoring structure is one of a pigtail, a J curve, and a flange.

In another aspect, a method for providing access to an interior of a human patient heart for a right ventricular assist procedure or an artificial lung procedure is described. The method includes providing a dual lumen cannula as described above, and passing the dual lumen cannula through an incision or a puncture into a lumen of a jugular vein of the patient and therethrough into the interior of the heart.

In one embodiment, the method includes steps of placing an atraumatic introducer in an interior of the dual lumen cannula second lumen and advancing the dual lumen cannula and introducer through the jugular vein lumen into the interior of the heart right atrium. Next, the atraumatic introducer is withdrawn and the selectively curvable portion is caused to assume the predetermined angle or curvature whereby the second lumen is positioned within the pulmonary artery lumen. Then, at least one pump is operatively connected to the dual lumen cannula whereby oxygen-depleted blood is removed from an interior of the right atrium through the first lumen distal drainage aperture and oxygen-enriched blood is delivered to the interior of the pulmonary artery through the second lumen, thereby by-passing a heart right ventricle to perform a right ventricular assist function.

In another embodiment, the method includes steps of providing an incision or puncture in a heart septa whereby fluid communication is established between the right atrium and a left atrium of the heart. Next, an atraumatic introducer is placed in an interior of the dual lumen cannula second lumen and the dual lumen cannula and introducer are passed through the patient jugular vein lumen into the interior of the heart right atrium. After positioning the dual lumen cannula whereby the drainage aperture of the first lumen of the dual lumen cannula is positioned within the right atrium, the atraumatic introducer is withdrawn and the selectively curvable portion is caused to assume the predetermined angle or curvature whereby at least the distal infusion aperture of the second lumen is passed through the incision or puncture into an interior of the left atrium. The second lumen may include a distal anchoring structure for preventing withdrawal of the second lumen from the incision or puncture after deployment. At least one pump is operatively connected to the dual lumen cannula whereby oxygen-depleted blood is removed from an interior of the right atrium through the first lumen and oxygen-enriched blood is delivered to the interior of the left atrium through the second lumen to perform an artificial lung function.

These and other embodiments, aspects, advantages, and features of the present invention will be set forth in the description which follows, and in part will become apparent to those of ordinary skill in the art by reference to the following description of the invention and referenced drawings or by practice of the invention. The aspects, advantages, and features of the invention are realized and attained by means of the instrumentalities, procedures, and combinations particularly pointed out in the appended claims. Various patent and non-patent citations are discussed herein. Unless otherwise indicated, any such citations are specifically incorporated by reference in their entirety into the present disclosure.

### BRIEF DESCRIPTION OF THE DRAWINGS

The accompanying drawings incorporated in and forming a part of the specification, illustrate several aspects of the present invention, and together with the description serve to explain the principles of the invention. In the drawings:

FIG. 1A shows a side cross-sectional view of a selectively occludable conduit according to the present disclosure;

FIG. 1B shows a side cross-sectional view of a dual lumen cannula according to the present disclosure:

FIG. 1C shows a side cross-sectional view of a dual lumen cannula with an extendable infusion sleeve;

FIG. 1D shows an end cross-sectional view of an dual lumen cannula according to the present disclosure;

FIG. 1E shows a side cross-sectional view of an alternative embodiment of the dual lumen cannula of FIG. 1B;

FIG. 2A shows the dual lumen cannula of FIG. 1E with an introducer in place;

FIG. 2B shows the dual lumen cannula of FIG. 2A with the introducer introducer partially removed;

FIG. 2C shows the dual lumen cannula of FIG. 2C with the introducer removed;

FIG. 3 illustrates a percutaneous approach for placement of the dual lumen cannula of the present disclosure for an artificial lung function; and

FIG. 4 illustrates a percutaneous approach for placement of the dual lumen cannula of the present disclosure for a right ventricular assist procedure.

### DETAILED DESCRIPTION OF THE ILLUSTRATED EMBODIMENTS

In the following detailed description of the illustrated embodiments, reference is made to the accompanying drawings that form a part hereof, and in which is shown by way of illustration, specific embodiments in which the invention may be practiced. These embodiments are described in sufficient detail to enable those skilled in the art to practice the invention. Also, it is to be understood that other embodiments may be utilized and that process, reagent, software, and/or other changes may be made without departing from the scope of the present invention.

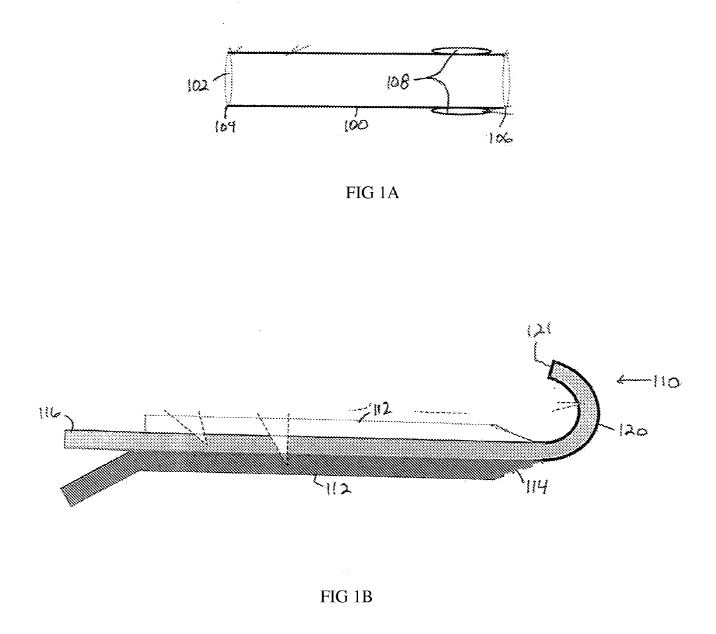

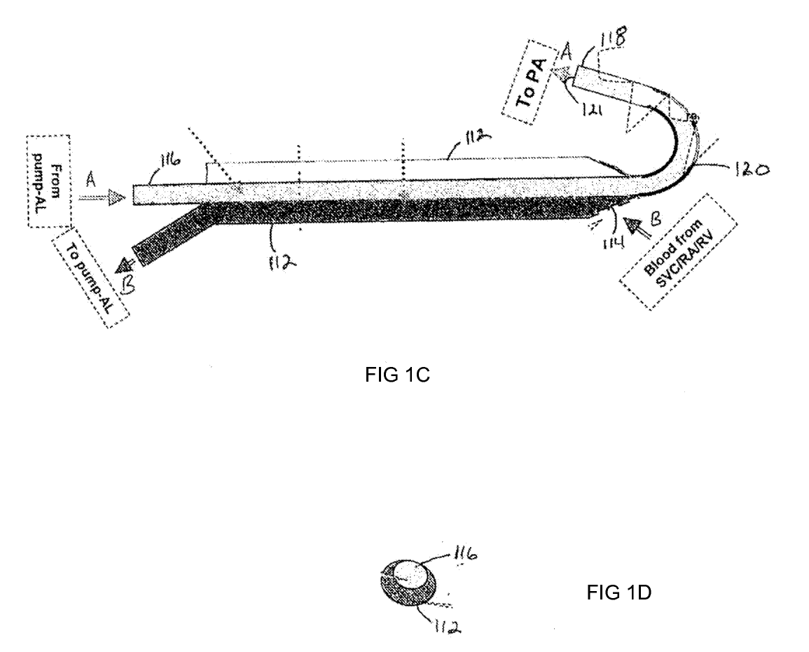

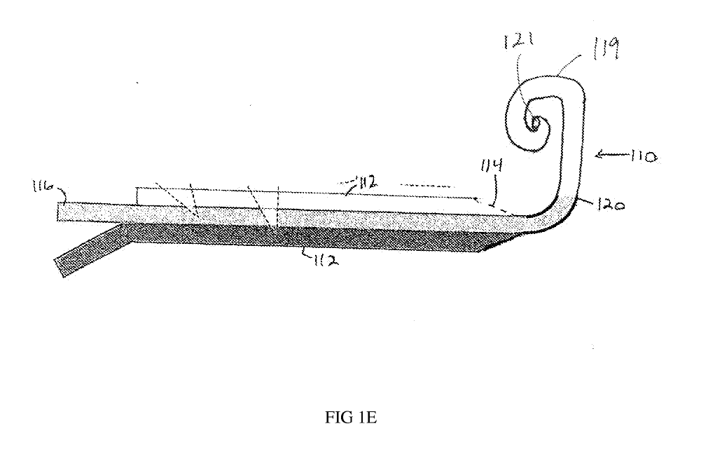

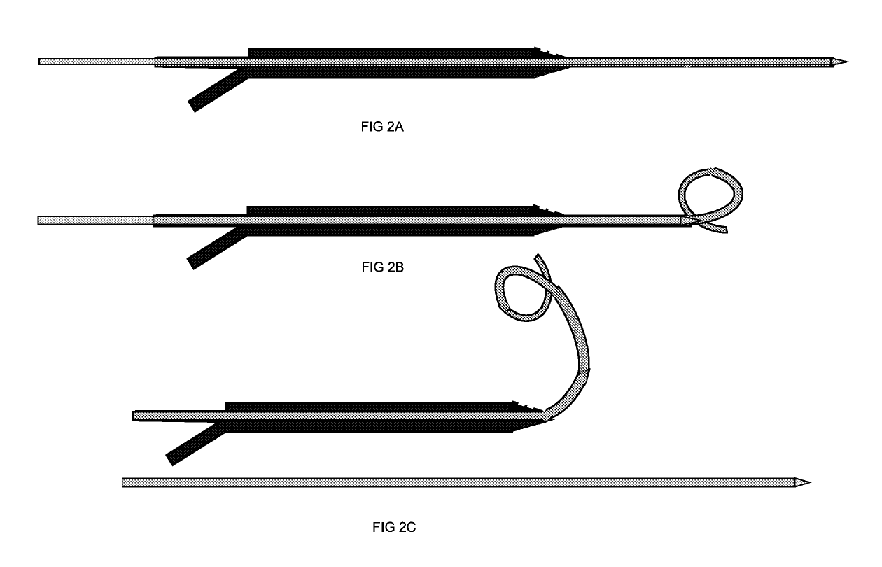

In one aspect, the present invention provides a system 10 (see FIGS. 1a-d) for providing access to a heart interior for delivering and/or removing a fluid. With reference to the drawings, FIG. 1A shows a selectively occludable conduit 100, defining an interior lumen 102 for providing access to an interior of at least one heart chamber (not shown in this figure) from an exterior of a chest wall of a patient (not shown in this Figure). The selectively occludable conduit 100 includes a first end 104 adapted to be anastomosed to the skin of a chest wall of the patient, and a second end 106 adapted to be anastomosed to the wall of a right atrium of the heart (not shown in this figure). As non-limiting examples, first end 104 and second end 106 may include one or more of suturing flanges and/or apertures (not shown for convenience) providing a plurality of attachment points for such anastomosis. The selectively occludable conduit 100 further includes an occlusion device such as an occlusion balloon 108, to substantially seal the end 106 of the conduit 100 anastomosed to the right atrium wall. It will be appreciated that this provides a mechanism for preventing bleeding and/or fluid leakage during use or between uses of the system 10.

The system 10 of the invention further includes a dual lumen cannula 110, adapted for passing through the interior lumen 102 of the conduit 100 to provide access to an interior of a heart, or for providing percutaneous access to an interior of the heart via a blood vessel, to allow delivering a fluid into and removing a fluid from the interior of the heart. Typically, the dual lumen cannula 110 defines a first, drainage lumen 112 for for establishing fluid communication with an interior of the heart for removing a fluid from the interior of the heart, and a second, delivery lumen 116 for establishing fluid communication with an interior of the heart for delivering a fluid into the interior of the heart.

The first lumen 112 is typically manufactured to provide a desired degree of stiffness to allow passing same through the conduit 100 described above, or through a lumen of a blood vessel, without sacrificing the required degree of flexibility. Any number of materials suitable for fabricating the first lumen 112 to provide the desired combination of flexibility and stiffness are known to the skilled artisan in this field. Still further, the first lumen 112 may include a surrounding reinforcing material or an integral reinforcing material (not shown for convenience) to provide the desired degree of stiffness and to prevent kinking. Suitable reinforcing materials which may be incorporated on or within the materials from which first lumen 112 is fabricated include without limitation wire, wire mesh, reinforcing polymers, and the like.

With reference to FIG. 1B, a draining end of first lumen 112 defines at least one drainage opening 114. The drainage opening 114 may optionally be sloped as shown in the drawing figure, to define a smooth transition portion between an outer surface of the first lumen 112 and an outer surface of the second lumen 116 for facilitating advancement of the cannula 110 through the above-described conduit 100 or through a lumen of a blood vessel (not shown). The skilled artisan will appreciate that the drainage opening 114 may define a wide range of angles from the vertical. The drainage opening 114 may be defined by an aperture, a perforated surface, or any structure suitable for allowing passage of a fluid to an interior of first lumen 112 for removal.

A translatable closure 115 (see FIG. 2) may be provided to occlude drainage opening 114 to substantially prevent entry of fluid into first lumen 112, such as during the process of advancing the dual lumen cannula 110 through the conduit 100 or through a blood vessel (not shown). The translatable closure 115 may be selectively translated from a closed to an open configuration (see arrow C) by any suitable means. As a non-limiting example, closure 115 may be hingedly connected to an end of drainage opening 114, whereby upon release of a catch or application of a suitable stimulus, closure 115 translates from a closed position occluding drainage opening 114 to an open position. Any number of suitable hinged connections 117 are contemplated, such as for example a flexibly polymer, spring steel, or the like. A wire or filament (not shown) may retain the closure 115 in the closed position. Once the dual lumen cannula 110 is advanced to the desired position (described in greater detail below), the wire or filament may be removed, allowing closure 115 to translate to the open position. Alternatively, the hinged connection 117 may be fabricated at least in part of a shape memory alloy, whereby application of a suitable stimulus such as an electrical current, saline held at a predetermined temperature, or the like, causes the shape memory alloy to contract, translating closure 115 to the open position.

In the depicted embodiment, the first lumen 112 and the second lumen 116 are arranged whereby at least a portion of the second lumen 116 is disposed in an interior of the first lumen 112. The second lumen 116 may be connected to the first lumen 112 along a length thereof, whereby at least a portion of the second lumen 116 is supported by the first lumen 112 (see FIG. 1D). Alternatively, the first lumen 112 and the second lumen 116 may be substantially coextensive along at least a portion of a longitudinal axis thereof, and separated by a sidewall or septum (not shown).

A distal end of the second lumen 116 terminates a distance beyond the draining end of the first lumen 112 at a distal infusion opening or aperture 121. The infusion aperture 121 may be defined by one or more apertures, a perforated surface, or any structure suitable for allowing passage of a fluid from an interior of second lumen 116 for infusion. In one embodiment of the invention, the second lumen 116 is provided with a selectively curvable portion 120 extending beyond the draining aperture 114 of the first lumen 112. This selectively curvable portion 120 may be induced to selectively assume a predetermined angle or curvature.

The skilled artisan will appreciate that any number of structures or mechanisms may be employed to provide that selectively curvable portion 120, and such structures or mechanisms are contemplated for use herein. For example, it is known to provide a spring elbow to create a predetermined angle or curvature in a lumen of a cannula, which elbow is kept in a straightened configuration by an atraumatic introducer 202 (see FIG. 2) sleeved in an interior of the cannula lumen. Upon removal of the introducer 202 from an interior of the lumen, the spring elbow assumes a predetermined shape or angle as shown in FIG. 1B. Such spring elbows may be manufactured of any suitable material, such as a suitably flexible polymer, spring steel or other suitably flexible metal, and the like. Similarly, it is known to provide a selectively flexible spring elbow (not shown in this figure) comprising a shape memory alloy such as nickel-titanium (NiTi). When kept at a first temperature, the NiTi sleeve remains in a substantially straightened position. However, upon exposure to a temperature change, such as by an electrical stimulus, body temperature, or sterile saline at an appropriate temperature, the NiTi sleeve or segment contracts to assume the predetermined shape or angle. Such devices for providing bends or predetermined angles/curvatures in cannulae are known in the art (see, for example, U.S. Pat. No. 7,090,659).

The predetermined angle created by the selectively curvable portion 120 is intended to alter a direction of flow of fluid passing through the second lumen 116 to a sufficient degree to deliver an end of the second lumen 116 including infusion aperture 121 into an interior of a pulmonary artery or a heart left atrium, allowing delivery of a fluid into that blood vessel while a fluid is removed from the heart right atrium via the first lumen 112. In embodiments, the selectively curvable portion 120 defines a length dimension that may dispose an infusion aperture 121 of the second lumen 116 at a distance of from about 4 cm to about 22 cm from the first lumen drainage aperture 114. These dimensions permit positioning an end of the first lumen 112 in the heart right atrium, and concurrently positioning an end of the second lumen 116 in a pulmonary artery lumen or the heart left atrium as will be described in detail below.

It will be appreciated that the drainage opening 114 may be positioned on a side of the dual lumen cannula opposed to the curvature of the selectively curvable portion 120 (see FIG. 1B) or may be positioned on a same side of the dual lumen cannula as the curvature of the selectively curvable portion 120 (see FIG. 1E). Advantageously, this latter configuration prevents inadvertent occlusion of the drainage opening 114 by, e.g., contact with a blood vessel lumen wall or heart chamber wall.

The second lumen 116 may optionally also include at least a portion which is fabricated at least in part from a suitable membrane material, a portion of which defines an infusion sleeve 118 for delivering a fluid such as oxygenated blood into an interior of the heart. That infusion sleeve 118 is extendable at least beyond the apertures 114 disposed at the draining end of the first lumen 112 (see FIG. 1C). In one embodiment, the extendable infusion sleeve 118 is simply a length of the suitable membrane material defining a lumen, which when not in use is disposed in an interior of the second lumen 116, such as in an interior of the selectively curvable portion 120, by rolling or folding therein. Fluid flow caused by passage of a fluid through the second lumen 116 causes the extendable infusion sleeve 118 to extend out of the selectively curvable portion 120. When positioned as described above, the extendable infusion sleeve 118 is dislodged by unrolling or unfolding, and extends into an interior of the blood vessel for delivery of the fluid thereinto.

In still other embodiments, second lumen 116 may also include an anchoring structure 119, known in this art for use to prevent inadvertent dislodging of the second lumen 116 from a surgical site once implanted. In one embodiment (See FIGS. 1E and 2A-2C), a so-called “pigtail” anchoring structure 119 is provided at an end of the second lumen 116, which provides a larger surface preventing dislodgement of the second lumen 116 such as through an incision. Of course, other structures are known including flanges, J curves, and others for preventing such dislodgement, and such structures are contemplated for use herein.

When coupled to one or more pumps and tubing as are known in this art, fluids such as oxygenated blood may be delivered through the second lumen, and fluids such as oxygen-depleted blood may be removed via the first lumen. Such operations will be described in greater detail below. Particular embodiments of the operations are described, including accessing the heart directly through the chest wall of a patient using the described selectively occludable conduit 100, and also percutaneous access to the heart via a blood vessel such as the jugular vein.

Of course, the skilled artisan will appreciate that the presently described dual lumen cannula 110 is suitable also for providing access to an interior of the heart via a percutaneous route, which may be more desirable in certain situations. As examples, it is known in a right atrium-pulmonary artery right heart assist procedure (RVAD) to place a cannula via the right jugular vein through the heart right atrium, heart right ventricle, to the pulmonary artery. In a right atrium-left atrium heart assist (artificial lung) procedure, it is known to place a cannula via the right jugular vein through the heart right atrium, across the heart septum dividing the heart right atrium and left atrium, and therethrough to the heart left atrium.

Specifically, the depicted dual lumen cannula 110, with an atraumatic introducer 202 sleeved therein, may be advanced through an incision or puncture providing access to a jugular vein of the body, and therethrough into an interior of the heart, wherein the drainage aperture 114 of the first lumen 112 is disposed within the right atrium 306 and substantially at a juncture of the superior vena cava 311, the right atrium 306, the right ventricle 312, and the inferior vena cava 313. The precise positioning of the dual lumen cannula 110 may be monitored by any acceptable method, including without limitation X-ray, ultrasound, etc.

The introducer 202 is removed, and the selectively curvable portion 120 is caused to assume the predetermined angle or curvature as described above, whereby the end of the second lumen 116 is disposed substantially at an opening of the pulmonary artery 314 and from there advanced into the pulmonary artery 314. Again, the desired positioning of the second lumen 116 may be visualized/confirmed by any suitable method. A fluid is then delivered through second lumen 116 into an interior of the pulmonary artery 314. This describes use of the dual lumen cannula 110 of the present invention in a right ventricular assist function, wherein the interior of the heart is accessed via a percutaneous route.

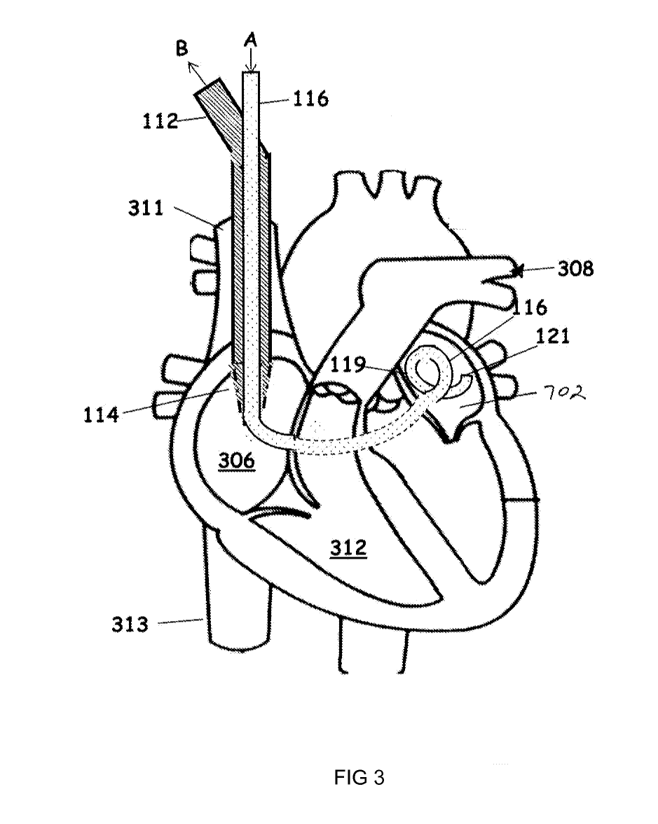

Similarly, an alternative embodiment of the dual lumen cannula 110 may be adapted for percutaneous access to an interior of the heart for an artificial lung function (see FIG. 3). The dual lumen cannula 110 having an atraumatic inducer 202 sleeved therein is passed through the jugular vein substantially as described above, with the exception that a dual lumen cannula 110 is provided having a second lumen 116 which is dimensioned whereby after the drainage opening 114 of the first lumen 112 is positioned within the right atrium 306 and substantially at a juncture of juncture of the superior vena cava 311, the right atrium 306, the right ventricle 312, and the inferior vena cava 313, the second lumen 116 infusion aperture 121 can be placed within the left atrium 702. As set forth above, a selectively curvable portion 120 having a length which positions the second lumen 116 infusion aperture 121 at a distance of from about 4 cm to about 22 cm from the first lumen drainage aperture 114 is contemplated.

The introducer 202 is removed, the selectively curvable portion 120 is caused to assume the predetermined angle or curvature as described, and the second lumen 116 is advanced into an interior of the left atrium 702 through an incision or puncture (not shown for convenience) traversing the atrial septum substantially as shown in FIG. 7. The anchoring structure 119 prevents inadvertent withdrawal of the second lumen 116 from the left atrium 702.

After operative connection of the dual lumen cannula 110 to one or more pumps in the manner described above, it will be appreciated that oxygen-depleted blood may be withdrawn via first lumen 112 from at least one of the superior vena cava 311, the right atrium 306, the right ventricle 312, and the inferior vena cava 313, and oxygenated blood may be delivered to the left atrium 702 via second lumen 116. Thus, an artificial lung function is established.

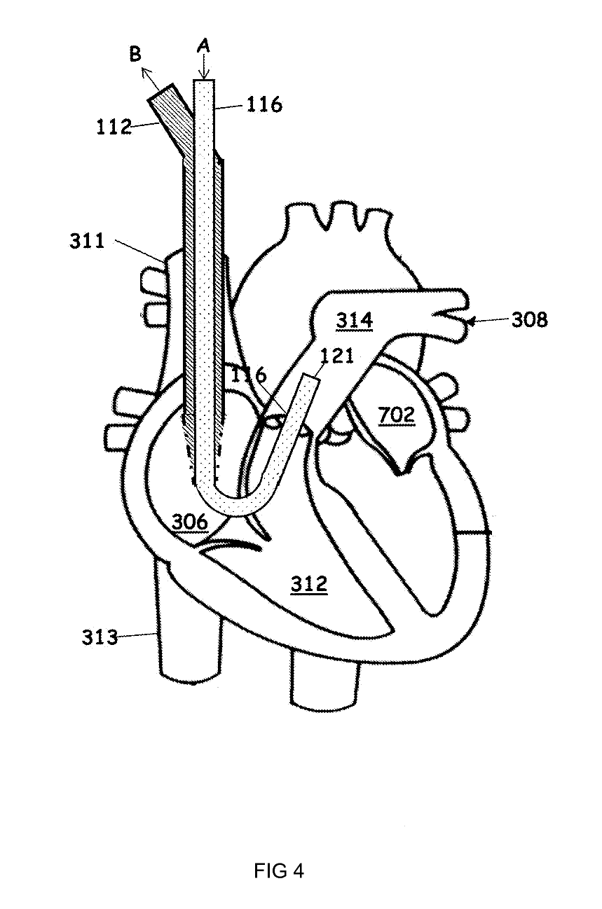

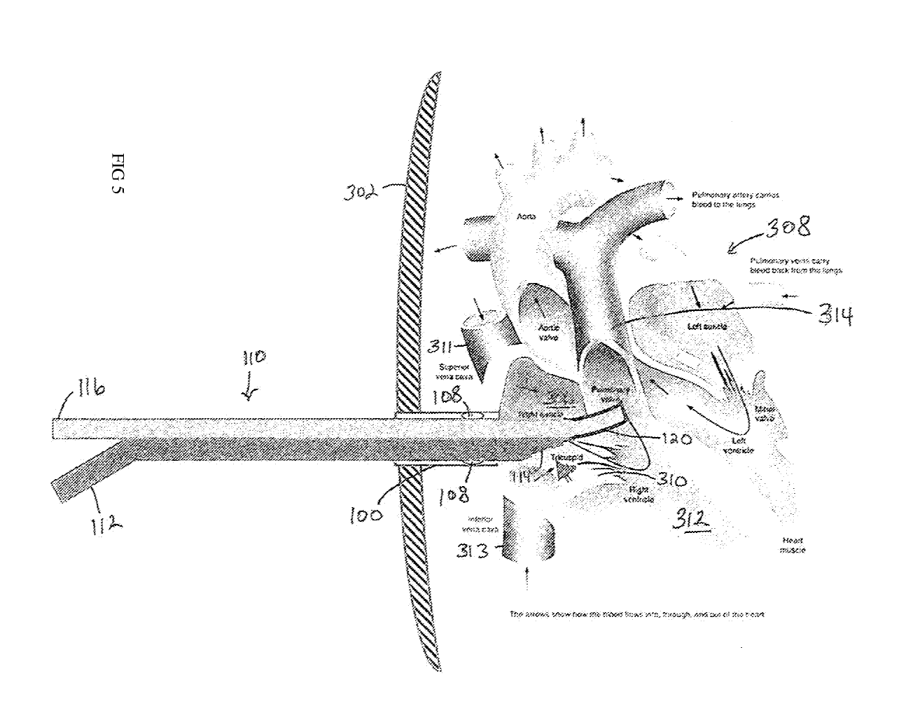

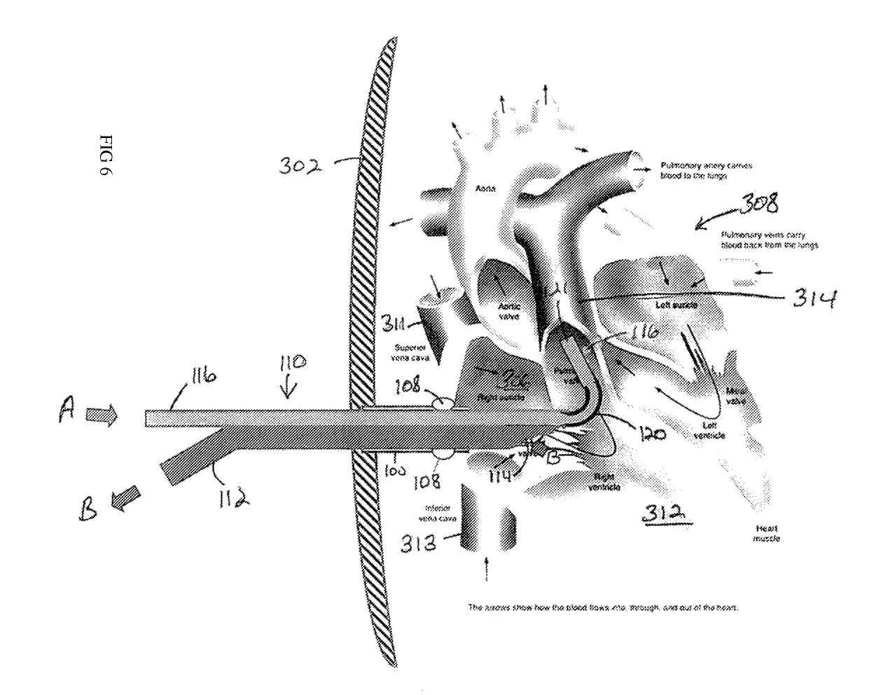

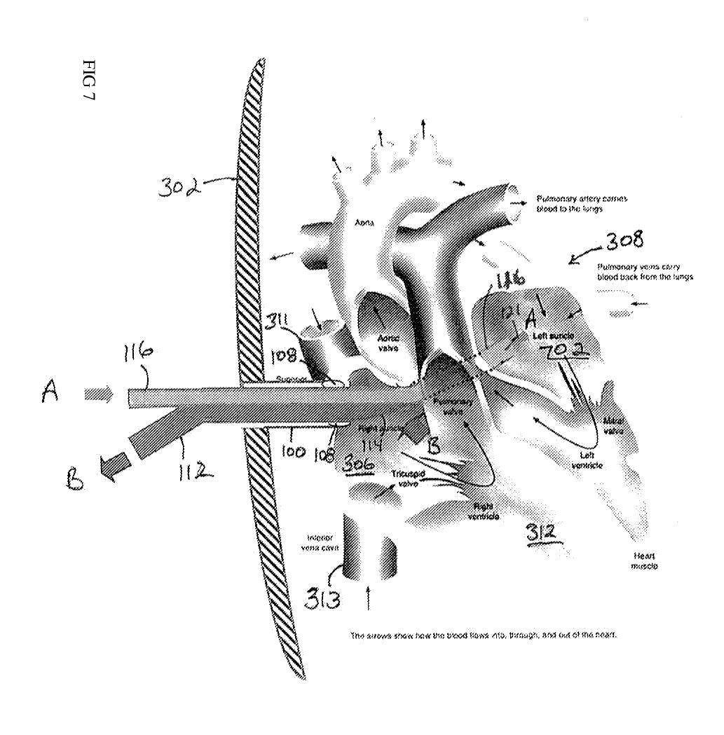

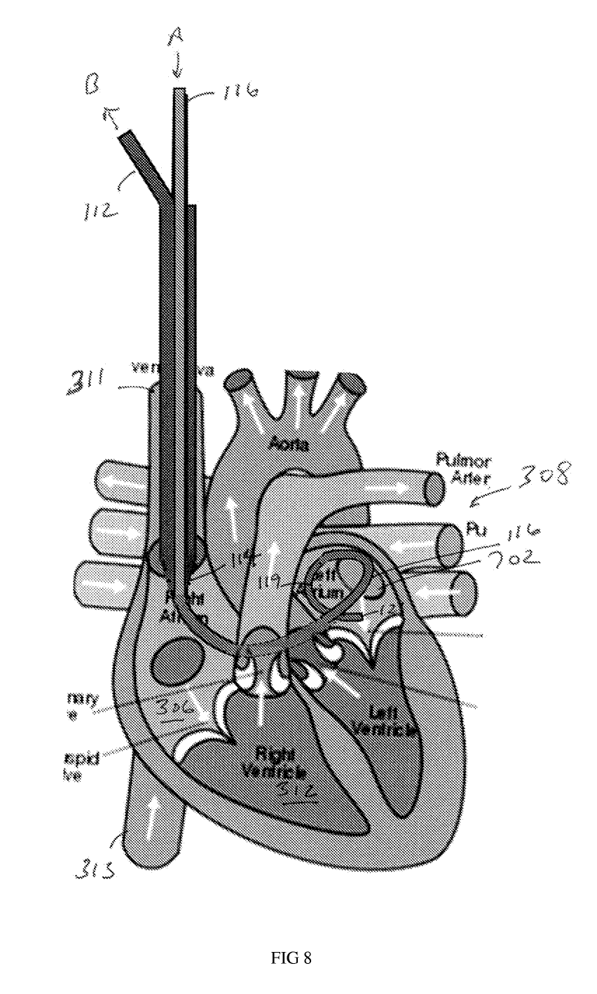

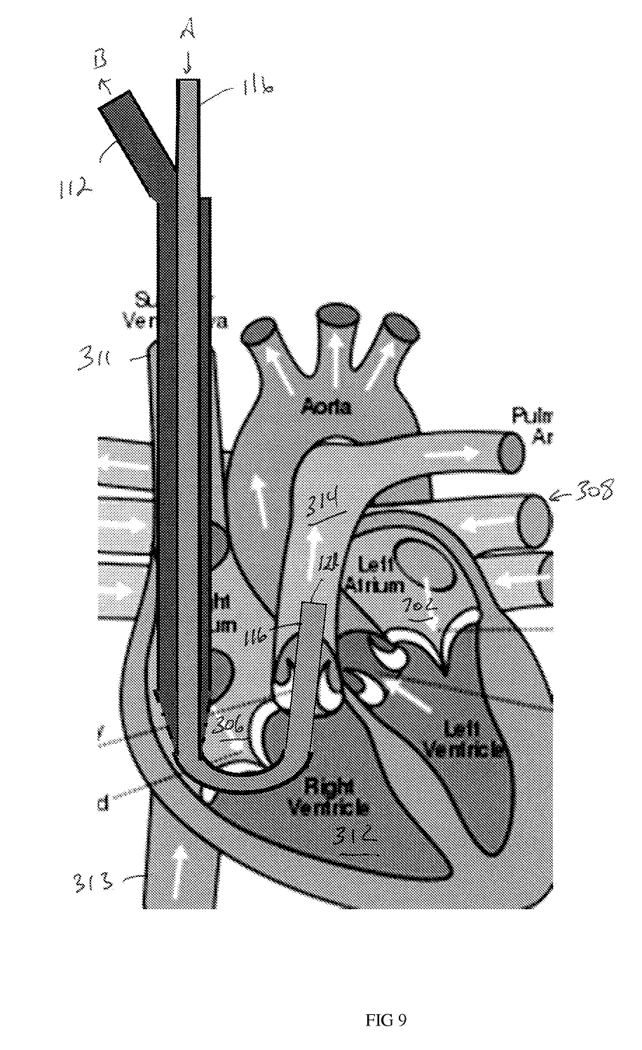

Likewise, yet another alternative embodiment of the dual lumen cannula 110 may be adapted for percutaneous access to an interior of the heart for an artificial lung function (see FIG. 4). The dual lumen cannula 110 having an atraumatic inducer 202 sleeved therein is passed through the jugular vein substantially as described above, with the exception that a dual lumen cannula 110 is provided having a second lumen 116 and a selectively curvable portion 120 which are dimensioned/configured whereby after the drainage opening 114 of the first lumen 112 is positioned within the right atrium 306 and substantially at a juncture of juncture of the superior vena cava 311, the right atrium 306, the right ventricle 312, and the inferior vena cava 313, the second lumen 116 infusion aperture 121 can be placed within the pulmonary artery 314.

The introducer 202 is removed, the selectively curvable portion 120 is caused to assume the predetermined angle or curvature as described, and the second lumen 116 is advanced into an interior of the pulmonary artery 314 as shown in FIG. 4.

After operative connection of the dual lumen cannula 110 to one or more pumps in the manner described above, it will be appreciated that oxygen-depleted blood may be withdrawn via first lumen 112 from at least one of the superior vena cava 311, the right atrium 306, the right ventricle 312, and the inferior vena cava 313, and oxygenated blood may be delivered to the pulmonary artery 314 via second lumen 116. Thus, a right ventricular assist function is achieved.

There is accordingly provided a system 10 for providing access to an interior of a patient's heart which allows repeated access for procedures such as right ventricular assist and artificial lung procedures, and which may be incorporated into devices and systems for such procedures. One of ordinary skill in the art will recognize that additional embodiments of the invention are also possible without departing from the teachings herein. This detailed description, and particularly the specific details of the exemplary embodiments, is given primarily for clarity of understanding, and no unnecessary limitations are to be imported, for modifications will become obvious to those skilled in the art upon reading this disclosure and may be made without departing from the spirit or scope of the invention. Relatively apparent modifications, of course, include combining the various features of one or more figures or examples with the features of one or more of other figures or examples.

What is claimed is:

1. A dual lumen cannula, comprising:
a first lumen having a first length and including at least one distal drainage aperture; and
a second lumen having a length that is greater than the first lumen length, a selectively curvable portion for assuming a predetermined angle or curvature, and a distal infusion aperture;
wherein the second lumen distal infusion aperture is disposed at a distance of from about 4 cm to about 22 cm from the first lumen distal drainage aperture to allow positioning an end thereof in an interior of a pulmonary artery lumen or a left atrium of the human heart when the first lumen distal drainage aperture is positioned within a right atrium of the human heart.

2. The dual lumen cannula of claim 1, further including a distal drainage aperture closure that is selectively translatable from a first, closed position to a second, open position.

3. The dual lumen cannula of claim 1, wherein the predetermined angle or curvature in the selectively curvable portion is created by at least one of a spring elbow, a flexible polymer, or a shape memory alloy.

4. The dual lumen cannula of claim 1, wherein the second lumen further includes a distal anchoring structure for preventing inadvertent withdrawal of the second lumen end from a surgical site.

5. The dual lumen cannula of claim 4, wherein the anchoring structure is one of a pigtail, a J curve, and a flange.

6. A method for providing access to an interior of a human patient heart for a right ventricular assist procedure or an artificial lung procedure, comprising:
providing a dual lumen cannula including a first lumen having a first length and including at least one distal drainage aperture and a second lumen having a second length for delivering a fluid into an interior of a human pulmonary artery lumen or an interior of a human heart left atrium, wherein the first length is less than the second length and further wherein the second lumen includes a selectively curvable portion for providing a predetermined angle or curvature and a distal infusion aperture, said second lumen distal infusion aperture being disposed at a distance of from about 4 cm to about 22 cm from the first lumen distal drainage aperture to allow positioning an end thereof within the interior of the pulmonary artery lumen or the human heart left atrium interior when the first lumen distal drainage aperture is positioned within a right atrium of the human heart; and
passing the dual lumen cannula through an incision or a puncture into a lumen of a jugular vein of the human patient and therethrough into the interior of the heart.

7. The method of claim 6, including providing a dual lumen cannula including a closure for substantially occluding the distal drainage aperture, the closure being selectively translatable from a first, closed position to a second, open position.

8. The method of claim 6, including providing a dual lumen cannula wherein the second lumen has a distal anchoring structure for preventing inadvertent withdrawal from a surgical site.

9. The method of claim 8, wherein the anchoring structure is one of a pigtail, a J curve, and a flange.

10. The method of claim 6, including the steps of:
placing an atraumatic introducer in an interior of the dual lumen cannula second lumen and advancing the dual lumen cannula and introducer through the jugular vein lumen into the interior of the heart right atrium;
withdrawing the atraumatic introducer and causing the selectively curvable portion to assume the predetermined angle or curvature whereby the second lumen is positioned within the pulmonary artery lumen; and
operatively connecting at least one pump to the dual lumen cannula whereby oxygen-depleted blood is removed from an interior of the right atrium through the first lumen distal drainage aperture and oxygen-enriched blood is delivered to the interior of the pulmonary artery through the second lumen, thereby by-passing a heart right ventricle to perform a right ventricular assist function.

11. The method of claim 6, including the steps of:
providing an incision or puncture in a heart septa whereby fluid communication is established between the right atrium and a left atrium of the heart;
placing an atraumatic introducer in an interior of the dual lumen cannula second lumen and passing the dual lumen cannula and introducer through the jugular vein lumen into the interior of the heart right atrium;
positioning the dual lumen cannula whereby the drainage aperture of the first lumen of the dual lumen cannula is positioned within the right atrium;
withdrawing the atraumatic introducer and causing the selectively curvable portion to assume the predetermined angle or curvature whereby at least a distal end of the second lumen is passed through the incision or puncture into an interior of the left atrium; and
operatively connecting at least one pump to the dual lumen cannula whereby oxygen-depleted blood is removed from an interior of the right atrium through the first lumen and oxygen-enriched blood is delivered to the interior of the left atrium through the second lumen to perform an artificial lung function.

12. The method of claim 11, including providing a dual lumen cannula including a second lumen having a distal anchoring structure for preventing inadvertent withdrawal of the second lumen from the incision or puncture in the heart septa.

13. The method of claim 11, wherein the anchoring structure is one of a pigtail, a J curve, and a flange.
